(19) United States
(12) Patent Application Publication
(10) Pub. No.: US 2014/0276846 A1
(43) Pub. Date: Sep. 18, 2014

(54) SYSTEMS AND METHODS FOR IMPLANTING BONE GRAFT AND IMPLANT

(71) Applicants: Richard G. MAULDIN, Erie, CO (US); Scott A. YERBY, Montara, CA (US); Paul SAND, Redwood City, CA (US); Bret W. SCHNEIDER, Morgan Hill, CA (US)

(72) Inventors: Richard G. MAULDIN, Erie, CO (US); Scott A. YERBY, Montara, CA (US); Paul SAND, Redwood City, CA (US); Bret W. SCHNEIDER, Morgan Hill, CA (US)

(21) Appl. No.: 14/216,790

(22) Filed: Mar. 17, 2014

Related U.S. Application Data
(60) Provisional application No. 61/793,357, filed on Mar. 15, 2013.

(51) Int. Cl. A61B 17/16

(52) U.S. Cl. CPC A61B 17/1664
USPC 606/80

(57) ABSTRACT

This application relates generally to broaches. More specifically, this application relates to broaches used to shape bores in bone. The broaches can shape the bores to receive an implant and also cut additional tubes or channels for receiving bone graft material and/or biologic aids.

### CROSS REFERENCE TO RELATED APPLICATIONS

This application claims priority to U.S. Patent Provisional Application No. 61/793,357, filed Mar. 15, 2013, and titled “SYSTEMS AND METHODS FOR IMPLANTING BONE GRAFT AND IMPLANT,” which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference. For example, this application incorporates by reference in their entireties U.S. Patent Publication No. 2011/0087294 and U.S. Patent Publication No. 2011/0118785.

### FIELD

This application relates generally to broaches. More specifically, this application relates to broaches used to cut bone.

### BACKGROUND

Many types of hardware are available both for the fixation of bones that are fractured and for the fixation of bones that are to be fused (arthrodesed).

For example, the human hip girdle is made up of three large bones joined by three relatively immobile joints. One of the bones is called the sacrum and it lies at the bottom of the lumbar spine, where it connects with the L5 vertebra. The other two bones are commonly called “hip bones” and are technically referred to as the right ilium and-the left ilium. The sacrum connects with both hip bones at the sacroiliac joint (in shorthand, the SI-Joint).

The SI-Joint functions in the transmission of forces from the spine to the lower extremities, and vice-versa. The SI-Joint has been described as a pain generator for up to 22% of lower back pain.

To relieve pain generated from the SI Joint, sacroiliac joint fusion is typically indicated as surgical treatment, e.g., for degenerative sacroiliitis, inflammatory sacroiliitis, iatrogenic instability of the sacroiliac joint, osteitis condensans ilii, or traumatic fracture dislocation of the pelvis. Currently, screws and screws with plates are used for sacro-iliac fusion. At the same time the cartilage has to be removed from the “synovial joint” portion of the SI joint. This requires a large incision to approach the damaged, subluxed, dislocated, fractured, or degenerative joint.

An alternative implant that is not based on the screw design can also be used to fuse the SI-Joint. Such an implant can have a triangular cross-section, for example, as further described below. To insert the implant, a cavity can be formed into the bone, and the implant can then be inserted into the cavity using a tool such as an impactor.

To improve integration of the implant with the bone, bone graft material can be applied to the implant before insertion into the bore or during the implantation procedure. Therefore, it would be desirable to provide systems, devices and methods for incorporating bone graft materials with the implant at the implantation site.

In addition, some methods of implantation of the implant require one or more drilling steps to form the bone cavity for receiving the implant. To reduce the number of drilling steps and simplify the procedure, it would be desirable to provide a modified broach that can efficiently cut the bone cavity with less drilling.

### SUMMARY OF THE DISCLOSURE

This application relates generally to broaches. More specifically, this application relates to broaches used to shape bores in bone. The broaches can shape the bores to receive an implant and also cut additional tubes or channels for receiving bone graft material and/or biologic aids.

In general, in one embodiment, a broach for shaping a bore in bone to receive an implant includes an elongate body with a proximal end, a distal end, at least three faces between the distal end and the proximal end, a plurality of apices formed at the junctions between adjacent faces, and a longitudinal axis. A lumen extends throughout the elongate body about the longitudinal axis, and the lumen is sized and shaped for receiving a guide pin. A plurality of cutting surfaces are located on the distal end of the elongate body for shaping the bore to receive the implant, and the plurality of cutting surfaces are oriented along the plurality of apices and become progressively smaller in size towards the distal end. A plurality of additional cutting surfaces is aligned with the plurality of apices for cutting channels in the bore to receive a bone graft material.

This and other embodiments can include one or more of the following features. Each face of the elongate body can include a channel extending along at least a portion of the longitudinal length of the elongate body. The elongate body can include three faces that define a substantially triangular cross-sectional profile transverse to the longitudinal axis. The plurality of cutting surfaces can be angled towards the distal end of the elongate body. The plurality of additional cutting surfaces can be partially circular. The plurality of additional cutting surfaces can be partially rectilinear.

In general, in one embodiment, a method for inserting an implant in bone includes: (1) drilling a bore into the bone; (2) inserting a broach to shape the bore to receive the implant and to form channels for receiving a bone graft material; (3) inserting the implant into the shaped bore; and (4) filling the channels with a bone graft material.

This and other embodiments can include one or more of the following features. The shaped bore can be rectilinear with a plurality of apices, and the channels can be formed at the apices of the shaped bore. The shaped bore can be triangular. The method can include inserting a guide pin into the bone. The steps of drilling a bore, inserting a broach, and inserting the implant all can be performed over the guide pin.

In general, in one embodiment, a broach for shaping a bore in bone to receive an implant includes an elongate body with a proximal end, a distal end, at least three faces between the distal end and the proximal end, a plurality of apices formed at the junctions between adjacent faces, and a longitudinal axis. A lumen extends throughout the elongate body about the longitudinal axis, and the lumen is sized and shaped for receiving a guide pin. A plurality of cutting surfaces is located on the distal end of the elongate body for shaping the bore to receive the implant. The plurality of cutting surfaces are oriented along the plurality of apices and become progressively smaller in size towards the distal end. A tapered distal tip portion at the distal end of the elongate body tapers to a distal opening of the lumen.

This and other embodiments can include one or more of the following features. The tapered distal tip portion can form a cutting surface around the opening of the lumen. The tapered distal tip portion can include a plurality of beveled faces that are angled towards the distal end. The tapered distal tip portion can include a smooth tapering surface that reaches the distal opening of the lumen. The elongate body can include three faces that define a substantially triangular cross-sectional profile transverse to the longitudinal axis. The plurality of cutting surfaces can be angled towards the distal end of the elongate body. Each face of the elongate body can include a channel extending along at least a portion of the longitudinal length of the elongate body.

In general, in one embodiment, a method for inserting an implant in bone includes: (1) inserting a guide pin into the bone; (2) inserting a sharp tipped broach over the guide pin to create a cavity for receiving the implant, wherein the cavity can be formed without first drilling a bore into the bone over the guide pin; and (3) inserting the implant into the cavity.

This and other embodiments can include one or more of the following features. The step of inserting a sharp tipped broach over the guide pin to create a cavity can include cutting the bone adjacent to the guide pine with one or more cutting edges at a distal end of the sharp tipped broach, and driving the sharp tipped broach further into the bone until a plurality of cutting surfaces on the sharp tipped broach can cut into and remove the bone surrounding the guide pin to form the cavity.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 illustrates an embodiment of an implant structure.

FIGS. 2A-2D are side section views of the formation of a broached bore in bone according to one embodiment of the invention.

FIGS. 2E and 2F illustrate the assembly of a soft tissue protector system for placement over a guide wire.

FIGS. 3 and 4 are, respectively, anterior and posterior anatomic views of the human hip girdle comprising the sacrum and the hip bones (the right ilium, and the left ilium), the sacrum being connected with both hip bones at the sacroiliac joint (in shorthand, the SI-Joint).

FIGS. 5 to 7A and 7B are anatomic views showing, respectively, a pre-implanted perspective, implanted perspective, implanted anterior view, and implanted cranio-caudal section view, the implantation of three implant structures for the fixation of the SI-Joint using a lateral approach through the ilium, the SI-Joint, and into the sacrum.

FIGS. 8A-8E illustrate embodiments of a modified broach for removing additional bone from a bore so that a bone graft or other material can be added with the implant.

FIGS. 9A and 9B illustrate an embodiment of a standard broach with a flat distal face.

FIGS. 10A and 10B illustrate an embodiment of the broach with a pointed distal tip portion.

FIGS. 11A and 11B illustrate an embodiment of the broach with an additional cutting surface located at the distal end of the broach.

FIGS. 12A and 12B illustrate an embodiment of the broach with a pyramid shaped distal tip.

FIG. 13 illustrates a CT scan with haloing artifacts around the implant.

### DETAILED DESCRIPTION

Elongated, stem-like implant structures 20 like that shown in FIG. 1 make possible the fixation of the SI-Joint (shown in anterior and posterior views, respectively, in FIGS. 3 and 4) in a minimally invasive manner. These implant structures 20 can be effectively implanted through the use a lateral surgical approach. The procedure is desirably aided by conventional lateral, inlet, and outlet visualization techniques, e.g., using X-ray image intensifiers such as a C-arms or fluoroscopes to produce a live image feed, which is displayed on a TV screen.

In one embodiment of a lateral approach (see FIGS. 5, 6, and 7A/B), one or more implant structures 20 are introduced laterally through the ilium, the SI-Joint, and into the sacrum. This path and resulting placement of the implant structures 20 are best shown in FIGS. 6 and 7A/B. In the illustrated embodiment, three implant structures 20 are placed in this manner. Also in the illustrated embodiment, the implant structures 20 are rectilinear in cross section and triangular in this case, but it should be appreciated that implant structures 20 of other rectilinear cross sections can be used.

Before undertaking a lateral implantation procedure, the physician identifies the SI-Joint segments that are to be fixated or fused (arthrodesed) using, e.g., the Fortin finger test, thigh thrust, FABER, Gaenslen's, compression, distraction, and diagnostic SI joint injection.

Aided by lateral, inlet, and outlet C-arm views, and with the patient lying in a prone position, the physician aligns the greater sciatic notches and then the alae (using lateral visualization) to provide a true lateral position. A 3 cm incision is made starting aligned with the posterior cortex of the sacral canal, followed by blunt tissue separation to the ilium. From the lateral view, the guide pin 38 (with sleeve (not shown)) (e.g., a Steinmann Pin) is started resting on the ilium at a position inferior to the sacrum end plate and just anterior to the sacral canal. In the outlet view, the guide pin 38 should be parallel to the sacrum end plate at a shallow angle anterior (e.g., 15.degree. to 20.degree. off the floor, as FIG. 7A shows). In a lateral view, the guide pin 38 should be posterior to the sacrum anterior wall. In the outlet view, the guide pin 38 should be superior to the first sacral foramen and lateral of mid-line. This corresponds generally to the sequence shown diagrammatically in FIGS. 2A and 2B. A soft tissue protector (not shown) is desirably slipped over the guide pin 38 and firmly against the ilium before removing the guide pin sleeve (not shown).

Over the guide pin 38 (and through the soft tissue protector), the pilot bore 42 is drilled in the manner previously described, as is diagrammatically shown in FIG. 2C. The pilot bore 42 extends through the ilium, through the SI-Joint, and into the S1. The drill bit 40 is removed.

The shaped broach 44 is tapped into the pilot bore 42 over the guide pin 38 (and through the soft tissue protector) to create a broached bore 48 with the desired profile for the implant structure 20, which, in the illustrated embodiment, is triangular. This generally corresponds to the sequence shown diagrammatically in FIG. 2D. The triangular profile of the broached bore 48 is also shown in FIG. 5.

FIGS. 2E and 2F illustrate an embodiment of the assembly of a soft tissue protector or dilator or delivery sleeve 200 with a drill sleeve 202, a guide pin sleeve 204 and a handle 206. In some embodiments, the drill sleeve 202 and guide pin sleeve 204 can be inserted within the soft tissue protector 200 to form a soft tissue protector assembly 210 that can slide over the guide pin 208 until bony contact is achieved. The soft tissue protector 200 can be any one of the soft tissue protectors or dilators or delivery sleeves disclosed herein. In some embodiments, an expandable dilator or delivery sleeve 200 as disclosed herein can be used in place of a conventional soft tissue dilator. In the case of the expandable dilator, in some embodiments, the expandable dilator can be slid over the guide pin and then expanded before the drill sleeve 202 and/or guide pin sleeve 204 are inserted within the expandable dilator. In other embodiments, insertion of the drill sleeve 202 and/or guide pin sleeve 204 within the expandable dilator can be used to expand the expandable dilator.

In some embodiments, a dilator can be used to open a channel though the tissue prior to sliding the soft tissue protector assembly 210 over the guide pin. The dilator(s) can be placed over the guide pin, using for example a plurality of sequentially larger dilators or using an expandable dilator. After the channel has been formed through the tissue, the dilator(s) can be removed and the soft tissue protector assembly can be slid over the guide pin. In some embodiments, the expandable dilator can serve as a soft tissue protector after being expanded. For example, after expansion the drill sleeve and guide pin sleeve can be inserted into the expandable dilator.

As shown in FIGS. 5 and 6, a triangular implant structure 20 can be now tapped through the soft tissue protector over the guide pin 38 through the ilium, across the SI-Joint, and into the sacrum, until the proximal end of the implant structure 20 is flush against the lateral wall of the ilium (see also FIGS. 7A and 7B). The guide pin 38 and soft tissue protector are withdrawn, leaving the implant structure 20 residing in the broached passageway, flush with the lateral wall of the ilium (see FIGS. 7A and 7B). In the illustrated embodiment, two additional implant structures 20 are implanted in this manner, as FIG. 6 best shows. In other embodiments, the proximal ends of the implant structures 20 are left proud of the lateral wall of the ilium, such that they extend 1, 2, 3 or 4 mm outside of the ilium. This ensures that the implants 20 engage the hard cortical portion of the ilium rather than just the softer cancellous portion, through which they might migrate if there was no structural support from hard cortical bone. The hard cortical bone can also bear the loads or forces typically exerted on the bone by the implant 20.

The implant structures 20 are sized according to the local anatomy. For the SI-Joint, representative implant structures 20 can range in size, depending upon the local anatomy, from about 35 mm to about 60 mm in length, and about a 7 mm inscribed diameter (i.e. a triangle having a height of about 10.5 mm and a base of about 12 mm). The morphology of the local structures can be generally understood by medical professionals using textbooks of human skeletal anatomy along with their knowledge of the site and its disease or injury. The physician is also able to ascertain the dimensions of the implant structure 20 based upon prior analysis of the morphology of the targeted bone using, for example, plain film x-ray, fluoroscopic x-ray, or MRI or CT scanning.

Using a lateral approach, one or more implant structures 20 can be individually inserted in a minimally invasive fashion across the SI-Joint, as has been described. Conventional tissue access tools, obturators, cannulas, and/or drills can be used for this purpose. Alternatively, the novel tissue access tools described above and in co-pending U.S. application Ser. No. 61/609,043, titled “TISSUE DILATOR AND PROTECTER” and filed Mar. 9, 2012, which is hereby incorporated by reference in its entirety, can also be used. No joint preparation, removal of cartilage, or scraping are required before formation of the insertion path or insertion of the implant structures 20, so a minimally invasive insertion path sized approximately at or about the maximum outer diameter of the implant structures 20 can be formed.

The implant structures 20 can obviate the need for autologous bone graft material, additional pedicle screws and/or rods, hollow modular anchorage screws, cannulated compression screws, threaded cages within the joint, or fracture fixation screws. Still, in the physician's discretion, bone graft material and other fixation instrumentation can be used in combination with the implant structures 20.

In a representative procedure, one to six, or perhaps up to eight, implant structures 20 can be used, depending on the size of the patient and the size of the implant structures 20. After installation, the patient would be advised to prevent or reduce loading of the SI-Joint while fusion occurs. This could be about a six to twelve week period or more, depending on the health of the patient and his or her adherence to post-op protocol.

The implant structures 20 make possible surgical techniques that are less invasive than traditional open surgery with no extensive soft tissue stripping. The lateral approach to the SI-Joint provides a straightforward surgical approach that complements the minimally invasive surgical techniques. The profile and design of the implant structures 20 minimize or reduce rotation and micromotion. Rigid implant structures 20 made from titanium provide immediate post-op SI Joint stability. A bony in-growth region 24 comprising a porous plasma spray coating with irregular surface supports stable bone fixation/fusion. The implant structures 20 and surgical approaches make possible the placement of larger fusion surface areas designed to maximize post-surgical weight bearing capacity and provide a biomechanically rigorous implant designed specifically to stabilize the heavily loaded SI-Joint.

To improve the stability and weight bearing capacity of the implant, the implant can be inserted across three or more cortical walls. For example, after insertion the implant can traverse two cortical walls of the ilium and at least one cortical wall of the sacrum. The cortical bone is much denser and stronger than cancellous bone and can better withstand the large stresses found in the SI-Joint. By crossing three or more cortical walls, the implant can spread the load across more load bearing structures, thereby reducing the amount of load borne by each structure. In addition, movement of the implant within the bone after implantation is reduced by providing structural support in three locations around the implant versus two locations.

In some embodiments, it may be desirable to add a bone graft material and/or biologic aid along with the implant in order to promote bone growth around and/or into the implant. An embodiment of a modified broach 800 is illustrated in FIGS. 8A and 8B. The modified broach 800 can be used in place of the broach 44 illustrated in FIG. 2D to create a shaped bore with channels for receiving a bone graft material and/or biologic aid.

The modified broach 800 can have a cross-sectional profile that generally matches the shape of the implant. For example, for a triangular shaped implant, the modified broach 800 can have a generally triangular shaped cross-sectional profile. Likewise, for an implant with a rectangular, square, or any other rectilinear shape, the modified broach 800 can have a generally matching cross-sectional profile. In some embodiments, as illustrated in FIG. 8B, the modified broach 800 has a generally triangular cross-sectional profile. The modified broach 800 can have a lumen or channel 802 extending along its entire longitudinal length and sized and shaped so that the modified broach 800 can be placed over a guide pin. The distal end 804 of the modified broach 800 can be tapered and have a plurality of cutting surfaces 806 that function to chisel away bone from the bore. The cutting surfaces 806 can be angled slightly towards the distal end 804 with the more proximal cutting surfaces 806 larger than the more distal cutting surfaces 806. In some embodiments, the cutting surfaces 806 are oriented with each apex of the modified broach 800. This configuration allows the modified broach 800 to progressively chisel away bone as the modified broach 800 is inserted into the bore. In some embodiments, the modified broach can also include one or more channels 808 that extend longitudinally along the sides of the modified broach 800 that aid in the removal of bone fragments from the bore. The channels 808 can be located along the center of each face of the modified broach 800, and can have a curved surface or be formed from two or more flat surfaces.

In some embodiments as illustrated in FIGS. 8A and 8B, the modified broach 800 can have additional cutting surfaces 810 located at each apex of the modified broach 800. In some embodiments, the additional cutting surfaces 810 can be located on one or more of the apices of the modified broach 800. In some embodiments, the additional cutting surfaces 810 can be located on each of the faces of the modified broach 800, such as where the channels 808 are shown in FIGS. 8A and 8B. In some embodiments as illustrated in FIGS. 8C-8E, the additional cutting surfaces 810, which can be circular, rectangular, triangular or any other suitable shape, can be located on one or more of the faces of the modified broach 800. In some embodiments, the additional cutting surfaces 810 can be located in a combination of one or more of the apices and faces of the modified broach 800. The additional cutting surfaces 810 can be angled slightly distally so that the cutting surfaces can chisel away bone fragments as the modified broach 800 is advanced into the bore. As described above, the additional cutting surfaces 810 can have circular shaped cutting surfaces or be any other shape, such as triangular, square, rectilinear, oval and the like. The channels can be sized to have a width or diameter of about 0.1 to 0.5 the width of a face or side of the bore.

The additional cutting surfaces 810 can cut tubes or channels from the shaped bore that can be filled bone graft material and/or a biologic aid. In some embodiments, the drilled bore can be enlarged using the modified broach 800 to shape the bore into a general shape that matches the implant while also cutting out bone graft channels that extend beyond the general implant profile. In some embodiments, the bone graft channels can be located at the apexes of the shaped bore.

In some embodiments, a standard broach can be used to shape the bore while additional tubes or channels can be made separately with a drill and specialized drill bit or drill fixture. In some embodiments, a standard broach can be used to initially shape the bore while a second broach can be used to cut out the additional tubes or channels.

As described above, the implant can be inserted into the shaped bore while bone graft material and/or a biologic aid can be inserted into the additional cut tubes or channels. In some embodiments, the bone graft material and/or biologic aids can be formed into solid rods, with shapes matching the cut tubes or channels, which can be impacted into each cut tube or channel. In other embodiments, the bone graft material and/or biologic aids can be injected with a specialized syringe or other injection device into each of the cut tubes or channels. In some embodiments, the bone graft material and/or biologic aids can also be smeared or coated onto the implant either before or as the implant in inserted into the shaped bore.

The bone graft materials can be a liquid, gel, slurry, paste, powder, solid structure, matrix of granular material or other form, and can include a biologic aid that can promote and/or enhance bony ingrowth, tissue repair, and/or reduce inflammation, infection and pain. For example, the bone graft materials and/or biologic aid can include growth factors, such as bone morphogenetic proteins (BMPs), hydroxyapatite in, for example, a liquid or slurry carrier, demineralized bone, morselized autograft or allograft bone, bone fragments, medications to reduce inflammation, infection or pain such as analgesics, antibiotics and steroids. In addition, a blood pellet formed by centrifugation of the patient's blood, for example, can be included in the bone graft materials. In some embodiments, the blood pellet can be added in pellet form to the bone graft materials, while in other embodiments, the blood pellet can be disassociated and mixed or incorporated with other bone graft materials and/or biologic aids. In some embodiments, the growth factors can be human recombinant growth factors, such as hr-BMP-2 and/or hr-BMP-7, or any other human recombinant form of BMP, for example. The carrier for the biologic aid can be a liquid or gel such as saline or a collagen gel, for example. The biologic aid can also be encapsulated or incorporated in a controlled released formulation so that the biologic aid is released to the patient at the implant site over a longer duration. For example, the controlled release formulation can be configured to release the biologic aid over the course of days or weeks or months, and can be configured to release the biologic aid over the estimated time it would take for the implant site to heal. The amount of biologic aid delivered to the implant structure can be controlled using a variety of techniques, such as controlling or varying the amount of coating material applied to the implant and/or controlling or varying the amount of biologic aid incorporated into the coating material. In some embodiments, in may be important to control the amount of biologic aid delivered because excessive use of certain biologic aids can result in negative effects such as radicular pain, for example.

In some embodiments, the filling of the cutting tubes or channels with bone graft material at the apices around the implant helps reduce haloing artifacts around the implant. As shown in FIG. 13, haloing refers to CT imaging artifacts 1300 that generally occur around corners of the implant 20 which can cause confusion in interpreting the CT image. Replacing the relatively sharp corners and apices with circular channels or tube can help to reduce the haloing artifacts.

FIGS. 9A and 9B illustrate a broach 900 without the additional cutting surfaces for cutting out additional tubes or channels. The broach 900 can have a cross-sectional profile that generally matches the shape of the implant. For example, for a triangular shaped implant, the broach 900 can have a generally triangular shaped cross-sectional profile, as illustrated in FIGS. 9A and 9B. Likewise, for an implant with a rectangular, square, or any other rectilinear shape, the broach can have a generally matching cross-sectional profile. The broach 900 can have a lumen or channel 902 extending along its entire longitudinal length and sized and shaped so that the broach 900 can be placed over a guide pin. The distal end 904 of the broach 900 can be tapered and have a plurality of cutting surfaces 906 that function to chisel away bone from the bore. The cutting surfaces 906 can be angled slightly towards the distal end 904 with the more proximal cutting surfaces 906 larger than the more distal cutting surfaces 906. In some embodiments, the cutting surfaces 906 are oriented with each apex of the broach 900. This configuration allows the broach 900 to progressively chisel away bone as the broach 900 is inserted into the bore. In some embodiments, the broach 900 can also include one or more channels 908 that extend longitudinally along the sides of the broach 900 that aid in the removal of bone fragments from the bore. The channels 908 can be located along the center of each face of the broach 900, and can have a curved surface or be formed from two or more flat surfaces. In some embodiments, the distal face 905 of the distal end 904 can be flat or blunt and be shaped generally like a ring with cutouts along the perimeter for the channels 908.

FIGS. 10A and 10B illustrate another embodiment of a broach 1000 with a similar design to the broach illustrated in FIGS. 9A and 9B, except that the broach 1000 illustrated in FIGS. 10A and 10B has a distal end 1004 that tapers into a pointed or bullet shaped tip rather than a flat surface. Like the broach 900 illustrated in FIGS. 9A and 9B, the broach 1000 illustrated in FIGS. 10A and 10B can have a cross-sectional profile that generally matches the shape of the implant. For example, for a triangular shaped implant, the broach 1000 can have a generally triangular shaped cross-sectional profile, as illustrated in FIGS. 10A and 10B. Likewise, for an implant with a rectangular, square, or any other rectilinear shape, the broach can have a generally matching cross-sectional profile. The broach 1000 can have a lumen or channel 1002 extending along its entire longitudinal length and sized and shaped so that the broach 1000 can be placed over a guide pin. The distal end 1004 of the broach 1000 can be tapered and have a plurality of cutting surfaces 1006 that function to chisel away bone from the bore. The cutting surfaces 1006 can be angled slightly towards the distal end 1004 with the more proximal cutting surfaces 1006 larger than the more distal cutting surfaces 1006. In some embodiments, the cutting surfaces 1006 are oriented with each apex of the broach 1000. This configuration allows the broach 1000 to progressively chisel away bone as the broach 1000 is inserted into the bore. In some embodiments, the broach 1000 can also include one or more channels 1008 that extend longitudinally along the sides of the broach 1000 that aid in the removal of bone fragments from the bore. The channels 1008 can be located along the center of each face of the broach 1000, and can have a curved surface or be formed from two or more flat surfaces. The proximal portion of the broach shaft 1010 can have markings 1012 that can provide indicators to the operator regarding the depth of penetration of the broach 1000 into the bone. The markings 1012 can be a transverse line and can include numerical indications of penetration depth.

However, in contrast to the embodiment of the broach illustrated in FIGS. 9A and 9B, the embodiment of the broach 1000 illustrated in FIGS. 10A and 10B has a pointed tip 1005 with a diameter at the distal end that is equal to the diameter of the lumen or channel 1002, and the diameter of the pointed tip 1005 can gradually increase in the proximal direction. The pointed tip 1005 can comprise a plurality of beveled faces 1009 angled towards the distal end 1004. The distal portion of the pointed tip 1005 can be formed into a smooth tapering surface 1007 that narrows until it reaches the lumen or channel 1002 at the distal end 1004. The smooth tapering surface 1007 can act as a cutting surface around the opening of the lumen 1002 to remove bone around the guide pin. As the broach 1000 traverses over the guide pin and is forced into the bone, the pointed tip 1005 can penetrate into the bone around the guide pin until the cutting surfaces 1006 can engage and chisel away the bone around the guide pin. Such a design can reduce or eliminate the need for additional drilling after the guide pin is place in the bone. The broach 1000 can be simply placed over the guide pin to foam the bore into the bone without the need of placing a drill bit over the guide pin and drilling a bore and then using the broach to shape the circular bore into a triangular or rectilinear bore.

FIGS. 11A and 11B illustrate another embodiment of a broach 1100 with a similar design to the broach illustrated in FIGS. 9A and 9B, except that the broach 1100 illustrated in FIGS. 11A and 11B has a distal end 1104 with an additional distal cutting surface 1103 adjacent to and surrounding the opening of the lumen or channel 1102 that forms the most distal part of the broach 1100. Like the broach 900 illustrated in FIGS. 9A and 9B, the broach 1100 illustrated in FIGS. 11A and 11B can have a cross-sectional profile that generally matches the shape of the implant. For example, for a triangular shaped implant, the broach 1100 can have a generally triangular shaped cross-sectional profile, as illustrated in FIGS. 11A and 11B. Likewise, for an implant with a rectangular, square, or any other rectilinear shape, the broach can have a generally matching cross-sectional profile. The broach 1100 can have a lumen or channel 1102 extending along its entire longitudinal length and sized and shaped so that the broach 1100 can be placed over a guide pin. The distal end 1104 of the broach 1100 can be tapered and have a plurality of cutting surfaces 1106 that function to chisel away bone from the bore. The cutting surfaces 1106 can be angled slightly towards the distal end 1104 with the more proximal cutting surfaces 1106 larger than the more distal cutting surfaces 1106. In some embodiments, the cutting surfaces 1106 are oriented with each apex of the broach 1100. This configuration allows the broach 1100 to progressively chisel away bone as the broach 1100 is inserted into the bore. In some embodiments, the broach 1100 can also include one or more channels 1108 that extend longitudinally along the sides of the broach 1100 that aid in the removal of bone fragments from the bore. The channels 1108 can be located along the center of each face of the broach 1100, and can have a curved surface or be formed from two or more flat surfaces. The proximal portion of the broach shaft 1110 can have markings 1112 that can provide indicators to the operator regarding the depth of penetration of the broach 1100 into the bone. The markings 1112 can be a transverse line and can include numerical indications of penetration depth.

However, as discussed briefly above, in contrast to the embodiment of the broach illustrated in FIGS. 9A and 9B, the embodiment of the broach 1100 illustrated in FIGS. 11A and 11B has a distal end 1104 with an additional distal cutting surface 1103 adjacent to and surrounding the opening of the lumen or channel 1102 that fauns the most distal part of the broach 1100. As the broach 1100 traverses over the guide pin and is forced into the bone, the distal cutting surface 1103 engages the bone around the guide pin and begins cutting, chiseling and removing the bone from around the guide pin, thereby starting the bore to receive the implant. As the broach 1100 penetrates further into the bone, the primary cutting surfaces 1106 can engage and chisel away additional bone around the guide pin, thereby enlarging the bore. Such a design can reduce or eliminate the need for additional drilling after the guide pin is place in the bone. The broach 1100 can be simply placed over the guide pin to form the bore into the bone without the need of placing a drill bit over the guide pin and drilling a bore and then using the broach to shape the circular bore into a triangular or rectilinear bore.

FIGS. 12A and 12B illustrate another embodiment of a broach 1200 having a pyramid shaped tip 1204. Like the broach 900 illustrated in FIGS. 9A and 9B, the broach 1200 illustrated in FIGS. 12A and 12B can have a cross-sectional profile that generally matches the shape of the implant. For example, for a triangular shaped implant, the broach 1200 can have a generally triangular shaped cross-sectional profile, as illustrated in FIGS. 12A and 12B. Likewise, for an implant with a rectangular, square, or any other rectilinear shape, the broach can have a generally matching cross-sectional profile. In some embodiments, the broach 1200 can have a lumen or channel 1202 extending along its entire longitudinal length and sized and shaped so that the broach 1200 can be placed over a guide pin.

The pyramid shaped tip 1204 can comprise three faces 1206 that taper towards the distal end of the broach 1200. At the distal end of the broach 1200 can be an opening to the lumen 1202. Surround the opening can be a plurality of cutting surfaces 1208, 1209 located at both the apices between the faces 1206 and along the distal end of each face 1206 between the apices. The cutting surfaces 1208, 1209 are configured to cut and chisel out the bone around the guide pin to form the bore for the implant. Furthermore, the cutting surfaces 1208 located at the apices can be arranged to form teeth with a pointed tip that can penetrate into and cut and chisel the bone surrounding the guide pin.

Variations and modifications of the devices and methods disclosed herein will be readily apparent to persons skilled in the art. As such, it should be understood that the foregoing detailed description and the accompanying illustrations, are made for purposes of clarity and understanding, and are not intended to limit the scope of the invention, which is defined by the claims appended hereto. Any feature described in any one embodiment described herein can be combined with any other feature of any of the other embodiment whether preferred or not.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1. A broach for shaping a bore in bone to receive an implant, the broach comprising:
an elongate body with a proximal end, a distal end, at least three faces between the distal end and the proximal end, a plurality of apices formed at the junctions between adjacent faces, and a longitudinal axis;
a plurality of cutting surfaces located on the distal end of the elongate body for shaping the bore to receive the implant, wherein the plurality of cutting surfaces are oriented along the plurality of apices and become progressively smaller in size towards the distal end; and
a plurality of additional cutting surfaces aligned with the plurality of apices for cutting channels in the bore to receive a bone graft material.

2. The broach of claim 1, wherein each face of the elongate body comprises a channel extending along at least a portion of the longitudinal length of the elongate body.

3. The broach of claim 1, wherein the elongate body comprises three faces that define a substantially triangular cross-sectional profile transverse to the longitudinal axis.

4. The broach of claim 1, wherein the plurality of cutting surfaces are angled towards the distal end of the elongate body.

5. The broach of claim 1, wherein the plurality of additional cutting surfaces are partially circular.

6. The broach of claim 1, wherein the plurality of additional cutting surfaces are partially rectilinear.

7. A method for inserting an implant in bone, the method comprising:
drilling a bore into the bone;
inserting a broach to shape the bore to receive the implant and to form channels for receiving a bone graft material;
inserting the implant into the shaped bore; and
filling the channels with a bone graft material.

8. The method of claim 7, wherein the shaped bore is rectilinear with a plurality of apices, and the channels are formed at the apices of the shaped bore.

9. The method of claim 8, wherein the shaped bore is triangular.

10. The method of claim 7, inserting a guide pin into the bone.

11. The method of claim 10, wherein the steps of drilling a bore, inserting a broach, and inserting the implant all are performed over the guide pin.

12. A broach for shaping a bore in bone to receive an implant, the broach comprising:
an elongate body with a proximal end, a distal end, at least three faces between the distal end and the proximal end, a plurality of apices formed at the junctions between adjacent faces, and a longitudinal axis;
a plurality of cutting surfaces located on the distal end of the elongate body for shaping the bore to receive the implant, wherein the plurality of cutting surfaces are oriented along the plurality of apices and become progressively smaller in size towards the distal end; and
a tapered distal tip portion at the distal end of the elongate body that tapers to a distal opening of the lumen.

13. The broach of claim 12, wherein the tapered distal tip portion forms a cutting surface around the opening of the lumen.

14. The broach of claim 12, wherein the tapered distal tip portion comprises a plurality of beveled faces that are angled towards the distal end.

15. The broach of claim 12, wherein the tapered distal tip portion comprises a smooth tapering surface that reaches the distal opening of the lumen.

16. The broach of claim 12, wherein the elongate body comprises three faces that define a substantially triangular cross-sectional profile transverse to the longitudinal axis.

17. The broach of claim 12, wherein the plurality of cutting surfaces are angled towards the distal end of the elongate body.

18. The broach of claim 12, wherein each face of the elongate body comprises a channel extending along at least a portion of the longitudinal length of the elongate body.

19. A method for inserting an implant in bone, the method comprising:
inserting a guide pin into the bone;
inserting a sharp tipped broach over the guide pin to create a cavity for receiving the implant, wherein the cavity can be formed without first drilling a bore into the bone over the guide pin; and
inserting the implant into the cavity.

20. The method of claim 19, wherein the step of inserting a sharp tipped broach over the guide pin to create a cavity comprises:
cutting the bone adjacent to the guide pine with one or more cutting edges at a distal end of the sharp tipped broach; and
driving the sharp tipped broach further into the bone until a plurality of cutting surfaces on the sharp tipped broach can cut into and remove the bone surrounding the guide pin to form the cavity.

21. The broach of claim 1, further comprising a lumen extending throughout the elongate body about the longitudinal axis, wherein the lumen is sized and shaped for receiving a guide pin.

22. The broach of claim 12, further comprising a lumen extending throughout the elongate body about the longitudinal axis, wherein the lumen is sized and shaped for receiving a guide pin.
